(19) United States
(12) Patent Application Publication
(10) Pub. No.: US 2011/0130823 A1
(43) Pub. Date: Jun. 2, 2011

(54) STENT HAVING FUNCTION ELEMENTS

(75) Inventors: Bodo Gerold, Zellingen (DE); Dietmar Esperschidt, Fuerth (DE)

(73) Assignee: BIOTRONIK VI PATENT AG, Baar (CH)

(21) Appl. No.: 12/945,049

(22) Filed: Nov. 12, 2010

Related U.S. Application Data
(60) Provisional application No. 61/264,855, filed on Nov. 30, 2009.

(51) Int. Cl. A61F 2/82

(52) U.S. Cl. 623/1.15; 623/1.46

(57) ABSTRACT

An endoprosthesis, in particular an intraluminal endoprosthesis, including a tubular base body and at least one function element, the at least one function element also being tubular and being arranged on the base body in such a way that the at least one function element surrounds the base body at least partially and in at least partial areas, so that it is aligned concentrically with the base body.

### CROSS REFERENCE TO RELATED APPLICATIONS

This invention claims benefit of priority to U.S. provisional patent application Ser. No. 61/264,855, filed on Nov. 30, 2009; the contents of which are herein incorporated by reference in their entirety.

### FIELD OF INVENTION

The invention relates to an endoprosthesis, in particular an intraluminal endoprosthesis having function elements.

### BACKGROUND OF THE INVENTION

In modern implantation medicine, implants are increasingly being used to reopen and support hollow organs such as blood vessels, the ureter, bile ducts, the uterus and bronchi in the human body.

Implantation of stents has become established as one of the most effective therapeutic measures for treating vascular diseases. The purpose of stents is to assume a supporting function in a patient's hollow organs. Stents of a traditional design therefore have a base body, which has a plurality of circumferential supporting structures, e.g., including metallic struts which are initially in a compressed form for insertion in to the body and are widened at the site of application. One of the main areas of application of such stents is for permanently or temporarily dilating and maintaining the patency of vasoconstrictions, in particular constrictions (stenoses) of the coronary vessels. In addition, there are also known aneurysm stents, which offer a supporting function for a damaged vascular wall and/or sealing of intracerebral aneurysms, for example.

In a medical procedure, balloon dilatation is first performed for treatment of a stenosis in a blood vessel, and then a stent is inserted to prevent renewed occlusion of the dilated vessel.

In another method, the balloon dilatation is performed simultaneously with the placement of the stent. In yet another method, constriction of the blood vessel is eliminated merely by inserting a self-expanding stent.

The stent has a base body of an implant material. An implant material is a nonviable material, which is used for an application in medicine and interacts with biological systems. The basic prerequisites for use of a material as an implant material which comes in contact with the physical environment of the body when used as intended is its biocompatibility. The term “biocompatibility” is understood to be the ability of a material to induce an appropriate tissue reaction in a specific application. This includes an adaptation of the chemical, physical, biological and morphological surface properties of an implant to the recipient tissue with the goal of achieving a clinically desired interaction. The biocompatibility of the implant material also depends on the chronological course of the reaction of the biosystem into which it is implanted. Thus, relatively short-term irritation and inflammation occur and may lead to tissue changes. Biological systems thus react in different ways as a function of the properties of the implant material. According to the reaction of the biosystem, the implant materials may be subdivided into bioactive, bioinert and biodegradable/absorbable materials.

For monitoring during the process of implantation and for the placement of the stent, traditional stents have a marker including one or more radiopaque materials.

The document DE 103 17 241 A1 discloses a stent having a metallic radiolucent basic mesh and marker elements containing a radiopaque material. Recesses provided by cutting them out of webs of the basic mesh of the known stent then act as a carrier structure. These recesses are surrounded by a cover layer of silicon carbide into which marker elements are subsequently welded. Another possibility disclosed in this document, although it is expensive, is to manufacture the basic mesh completely of a Nitinol wire having a gold core which serves as an X-ray marker. This Nitinol wire, which has been provided with the gold core, forms the entire end section of the stent and is connected to the basic mesh by welding.

A similar approach is also described in the document DE 100 64 569 A1. According to the method disclosed in this document for applying a marker element to a stent, a free-flowing or pourable material or material mixture, e.g., in the form of granules, to be solidified is introduced into a recess in the basic mesh of the stent and solidified there. The solidification of this material is accomplished by sintering, for example.

To support the treatment, for some time now, stents have been coated with medications. These medications serve to prevent/reduce inflammatory responses of the vascular walls after the procedure (anti-inflammatory) or excessive proliferation of smooth vascular to muscle cells, which may lead to a restenosis. Many substances also act to accelerate colonization of the stent with endothelial cells. This desired effect accelerates the ingrowth of the stent into the vascular wall.

Arrangements of this type are known, for example, from U.S. Pat. No. 6,120,536, which discloses a coronary stent including a polymer coating into which heparin is incorporated and which optionally has an active-ingredient-free cover layer on the heparin-incorporating layer. In addition, there are also known coronary stents, which contain rapamycin in a nonabsorbable polymeric carrier matrix on the coronary stent.

All the approaches described here have the disadvantage that the additional functions, which should be offered by a stent in addition to maintaining the patency of a blood vessel, are associated fixedly with the stent. There is no possibility of varying the stent with its additional functions—such as the marker or medication coating.

### SUMMARY OF THE INVENTION

The object of the present invention is therefore to make available an endoprosthesis including a base body and one or more function elements. The function elements should be selectable from a variety of different function elements, so they can be coordinated with the various therapeutic concepts. At the same time, it should be possible to ensure that the behavior of the stent when used as intended—such as flexibility, recoil and supporting forces—is not altered in a deleterious manner.

This object is achieved by an endoprosthesis including a tubular base body and at least one function element. The at least one function element is also tubular and is arranged on the base body in such a way that it surrounds the base body in at least some partial areas and does so at least partially, so that it is arranged concentrically with the base body.

### DESCRIPTION OF THE DRAWINGS

FIG. 1 shows schematically the base body 10 of the endoprosthesis 1 in the form of a (basic) stent having a recess 12 according to an exemplary embodiment of the present invention.

FIG. 2 shows schematically a function element 20 according to the exemplary embodiment of FIG. 1.

FIG. 3 shows schematically the entire endoprosthesis 1 in the form of a stent system according to the exemplary embodiment of FIG. 1.

FIG. 4 shows the base body 10 and function element 20 in the view along the longitudinal axis A of the endoprosthesis 1 according to the exemplary embodiment of FIG. 1.

FIG. 5 also shows the base body 10 and the function element 20 in the view along the longitudinal axis A of the endoprosthesis 1 according to the exemplary embodiment of FIG. 1.

### DETAILED DESCRIPTION OF THE INVENTION

Both the base body and the function element(s) preferably include(s) a metallic material of one or more metals from the group including iron, magnesium, nickel, tungsten, titanium, zirconium, niobium, tantalum, zinc, silicon, lithium, sodium, potassium, calcium, manganese. In another exemplary embodiment, the base body and function elements include a memory material of one or more materials from the group including nickel-titanium alloys and copper-zinc-aluminum alloys, but preferably Nitinol. In another preferred exemplary embodiment, the base body and function elements are made of stainless steel, preferably a Cr—Ni—Fe steel—preferably the alloy 316L here—or a Co—Cr steel. the base body of the stent may also include at least partially a polymer (e.g., PLLA, PLGA, HA, PU) and/or a ceramic.

In a preferred embodiment, the base body and/or function elements include a biodegradable alloy selected from the group including magnesium, iron, zinc and tungsten. The biodegradable metallic material is a magnesium alloy in particular. The term alloy is understood in the present context to refer to a metallic structure whose main component is magnesium, iron, zinc or tungsten. The main component is the alloy component whose amount by weight in the alloy is the greatest. The main component preferably amounts to more than 50 wt %, in particular more than 70 wt %. If the material is a magnesium alloy, then it preferably contains yttrium and additional rare earth metals because such an alloy is excellent due to its physicochemical properties and high biocompatibility, in particular also its degradation products. The magnesium alloy WE43 is especially preferred.

Biodegradable alloys are those in which degradation takes place in a physiological environment, ultimately resulting in the entire endoprosthesis or the part of the endoprosthesis formed from the material losing its mechanical integrity.

Additional exemplary embodiments relate to the unlimited possibilities for a combination of degradable and nondegradable parts of the endoprosthesis. In one exemplary embodiment, the endoprosthesis is composed of a nondegradable base body and one or more degradable function elements. It is also conceivable for the endoprosthesis to include a nondegradable base body and multiple function elements, whereby individual function elements are biodegradable and other function elements are nondegradable. Nondegradable function elements may be, for example, X-ray markers which mark the treated site in the blood vessel even when the stent has long ago been degraded. This may be important for the patient's follow-up care.

Due to the inventive modular concept, it is possible to connect one or more function elements selectable from a larger number of different function elements to the base body. Due to the fact that the function elements are arranged concentrically around the base body, neither the diameter nor the circumference of the endoprosthesis is especially enlarged. At the same time the required flexibility of the endoprosthesis is not limited due to the type of arrangement.

In an especially preferred embodiment, the base body has recesses, which can be filled with a function element. Depending on the concept, there may be only one recess here which is filled with a function element. However, multiple recesses may also be provided on the base body and filled with the corresponding number of function elements. Due to the arrangement of the function elements in the recesses, an especially high flexibility of the endoprosthesis is ensured. At the same time, the circumference and/or diameter of the endoprosthesis is not enlarged with this approach. One or two recesses located closed to the ends of the endoprosthesis in the axial direction are especially preferred.

The base body or the function element or both preferably include a mesh structure. The mesh structure may have an open-celled or closed-cell design. The intraluminal endoprosthesis may be, for example, a stent system in which the base body is a (basic) stent on which the function element is arranged. The (basic) stent, i.e., the base body here, includes a mesh-like circumferential wall, which allows the (basic) stent to be inserted in a compressed (crimped) state with a small outside diameter as far as the site to be treated in the respective blood vessel and then to be dilated there, e.g., with the help of a dilation balloon catheter, until the blood vessel has the desired enlarged inside diameter. However, it may also be a self-expanding stent. Then both the base body and the function elements are made of a shape memory alloy, e.g., Nitinol, or they have a self-expanding stent design (e.g., wall stent and/or coil design).

The function element may also include a mesh structure, which in the case of application to a base body functioning as the (basic) stent, may have the same properties as the stent. This means that the function element may also be compressed (crimped) to a small outside diameter and dilated at the site of treatment.

Attaching the base body and function element to a balloon catheter, for example, is accomplished in two successive steps. In the first step, the base body is advanced over the balloon and crimped there. In a second step, the function element is brought over this arrangement of balloon and base body and then crimped. This system has the advantage that due to the exterior function element, which is also crimped with a certain pressure, the base body underneath is pressed more tightly against the balloon. The retention power of the endoprosthesis and/or the (basic) stent on the balloon is thus increased. The risk of slippage of the entire stent system on the balloon or even the risk of complete loss of the stent is thus greatly reduced.

However, simultaneous attachment and crimping of the base body and function element would also be conceivable. The base body and function element in uncrimped form are advanced over the balloon here. The function element in this case has a slightly larger diameter than the base body. When both parts of the endoprosthesis are in the correct position, they are crimped at the same time and thus the compressed form having a small outside diameter as required for implantation is imparted. The retention power of the endoprosthesis and/or the (basic) stent on the balloon is also increased in this case.

In a preferred embodiment, the base body and function element have the same mesh structure. For example, the base body may have one of the known helical and/or meandering stent patterns, and the function element receives this mesh structure. In this preferred embodiment, the base body may also have the recesses described above, which may be filled by one or more function elements. These recesses are preferably located at the ends of the endoprosthesis in the axial direction and may replace the next-to-last ring segment of the mesh structure, for example. This then yields a form-fitting endoprosthesis and/or a stent system having only slight overlapping of the structure and therefore being excellently compressible as well as especially flexible.

Overlapping of the base body and function elements can be compensated through thinner structures accordingly at the sites of the overlapping. Thus, for example, the mesh structure of the base body as well as the function elements may have thinner web widths at the sites of the overlapping.

Implantation of an endoprosthesis and/or the process of positioning and expansion of the stent system during the procedure, and the final position of the stent and the tissue after the end of the procedure must be monitored by the cardiologist. This can be done by means of imaging methods, e.g., by X-ray examinations. Therefore, in a preferred embodiment, the function element serves as a marker. To this end, the function element contains and/or includes a radiopaque material. Due to its shape, which circumscribes the entire circumference of the tubular structure of the base body, the marker covers a relatively large area and is especially clearly discernible with the known imaging methods.

For degradable endoprostheses, filled polymers (e.g., poly-L-lactide, poly-D,L-lactide, triblock copolymers, polyorthoesters, polysaccharides) or degradable metals and/or metal alloys (e.g., Fe, Zn, Mg, W, FeMn alloys, MgAl alloys such as AZ31, AZ80, AZ91; magnesium-rare-earth alloys such as WE43) are available. For nondegradable endoprostheses, the radiopaque material may be composed of one or more elements of the group including Ta, W, Au, Ir, Pt and alloys thereof.

In another preferred exemplary embodiment, the function element contains one or more active ingredients. The function element may be coated with one or more active ingredients or the material of the function element may be permeated with an active ingredient. An active ingredient in the sense of the present invention is a vegetable, animal or synthetic active pharmaceutical substance which is used in a suitable dosage as a therapeutic agent for influencing states or functions of the body, as a substitute for natural active ingredients produced by the human or animal body and for eliminating disease pathogens or exogenous substances or for rendering them harmless. Release of the substance in the environment of the implant has a positive effect on the course of healing and/or counteracts pathological changes in the tissue due to the surgical procedure.

Such active pharmaceutical substances have, for example, anti-inflammatory and/or antiproliferative and/or spasmolytic effects, so that restenoses, inflammations or (vascular) spasms, for example, can be prevented. In especially preferred exemplary embodiments, such substances may include one or more substances of the group of active ingredients including calcium channel blockers, lipid regulators (e.g., fibrates), immunosuppressants, calcineurin inhibitors (e.g., tacrolimus), antiphlogistics (e.g., cortisone or diclofenac), anti-inflammatories (e.g., imidazoles), anti-allergies, oligonucleotides (e.g., dODN), estrogens (e.g., genistein), endothelializing agents (e.g., fibrin), steroids, proteins, hormones, insulins, cytostatics, peptides, vasodilators (e.g., sartans), antiproliferative agents or taxols or taxans, here preferably paclitaxel or sirolimus and its derivatives as well as agents from lipophilic substances, which inhibit tissue calcification or formation of neointima, such as vitamin A and D derivatives and phylloquinone/menaquinone (vitamin K) derivatives.

According to another preferred embodiment, the endoprosthesis has one or more coatings. The base body and the function elements may be coated individually, separately from one another and with different materials; or the base body and function elements may have the same continuous coating. The coatings may each be biodegradable or permanent, i.e., nondegradable.

These may be coatings which are capable of absorbing or transporting the active ingredients, but the coatings may also have the function of protecting the endoprosthesis or parts thereof from abrasion, which may act on the endoprosthesis, whether due to handling of the endoprosthesis outside of the body, due to the implantation procedure or due to the physiological environment in the human or animal body. This coating may include parylene, Teflon, DLC, SiC, polyurethanes, polyesterimides, polyimides, for example, or it may be a coating obtained by nitration.

In addition, the coating may also be necessary or desired to suppress galvanic effects between two metallic components of the endoprosthesis. This may be the case, for example, with a base body containing magnesium and a function element containing gold. Coatings of, for example, parylene, Teflon, DLC, SiC, silicone, polyurethanes, polyesterimides and/or polyimides are available here.

Conversely, however, precisely this galvanic effect in the form of contact corrosion may be desired, so that a coating which provides cathodic protection for the base body, for example, by electrically bonding the function element(s) to the base body may be desired. For example, the base body may include titanium-zinc (a zinc alloy with a small amount of titanium (approx. 0.5-3%)) or an iron-manganese alloy. The function element(s) may include a magnesium alloy such as WE43, for example.

To absorb and/or transport active ingredients, coatings of one or more different polymers are expedient. Polymers from the group including the following components are preferred: (1) nondegradable (permanent) polymers, including polypropylene; polyethylene; polyvinyl chloride; polyacrylates, preferably polyethyl acrylates and polymethyl acrylates, polymethyl methacrylate; polymethyl-co-ethyl acrylate, ethylene/ethyl acrylate, etc.; polytetrafluoroethylene, preferably ethylene/chlorotrifluoroethylene copolymers, ethylene/tetrafluoroethylene copolymers; polyamides, preferably polyamidimide, PA-11, -12, -46, -66, etc.; polyetherimide; polyethersulfone; poly(iso)butylene; polyvinyl chloride; polyvinyl fluoride; polyvinyl alcohol; polyurethane; polybutylene terephthalate; silicones; polyphosphazene; polymer foams, preferably of carbonates, styrenes, etc. and copolymers and blends of the classes listed and/or the class of thermoplastics in general; and (2) biodegradable polymers, including polydioxanone; polyglycolide; polycaprolactone; polylactides, preferably poly-L-lactide, poly-D,L-lactide, and copolymers and blends such as poly(L-lactide-co-glycolide), poly(D,L-lactide-co-glycolide), poly(L-lactide-co-D,L-lactide), poly(L-lactide-co-trimethylene carbonate); triblock copolymers; polyorthoesters; polysaccharides, preferably chitosan, levan, hyaluronic acid, heparin, dextran, cellulose, chondroitin sulfate, etc.; polyhydroxyvalerate; ethylvinyl acetate; polyethylene oxide; polyphosphorylcholine; peptides, preferably fibrin, albumin, polyhydroxybutyric acid, preferably atactic, isotactic, syndiotactic and blends thereof.

Polylactides are especially preferred, in particular poly-L-lactide, poly-D,L-lactide, and copolymers as well as blends such as poly(L-lactide-co-glycolide), poly(D,L-lactide-co-glycolide), poly(L-lactide-co-D,L-lactide), poly(L-lactide-co-trimethylene carbonate), and polysaccharides, preferably chitosan, levan, hyaluronic acid, heparin, dextran, cellulose, chondroitin sulfate and polypeptides, preferably fibrin and albumin.

For special medical applications, it is possible to implement the coatings of the function elements with their incorporated active ingredients, so that the endoprosthesis has active ingredient kinetics tailored to the treatment of the patient. Thus, for example, a portion of the function elements may have coatings with active ingredients, in which it is desirable to have rapid kinetics, i.e., the active ingredients should be released during or a short time after implantation. Another portion of the function elements may combine the coating and active ingredient with slow kinetics, so the active ingredient is either released slowly or is released at a later point in time after implantation. For example, faster kinetics may be achieved by embedding the active ingredient (e.g., paclitaxel) in PLGA, and slower kinetics may be achieved by embedding the active ingredient in PLLA.

Production of the base body as well as the function elements may be accomplished in all the traditional ways: laser beam cutting, extrusion, welding, casting, bending, crimping, folding, riveting, hard soldering or soft soldering.

Crimping is also performed in the traditional manner. For accurate positioning of the base body and function elements, specially prepared positioning devices may be used.

The substantial advantage of the modular concept is the possibility of individual adaptation of the type of function elements to the requirements of the treatment. For example, the number and type of function elements which are attached to the base body, which is also individually selected, may be adapted to the particular patient and his symptoms, diseases, medical requirements, etc. It is possible to take into account allergies and intolerance conditions without any difficulty. Furthermore, the size, stature and constitution of the patient may be taken into account—if necessary, just shortly before the procedure—if, for example, the medical personnel is enabled to decide about the composition of the base body and the particular function elements required shortly before the surgical procedure and to assemble the endoprosthesis according to the modular principle.

At the same time, this modular concept is space-saving because it is not necessary to maintain a supply of all conceivable endoprostheses, but instead merely the individual parts which are taken as needed. For the same reasons, the inventive approach reduces costs for both the manufacturer and the consumer.

### Example

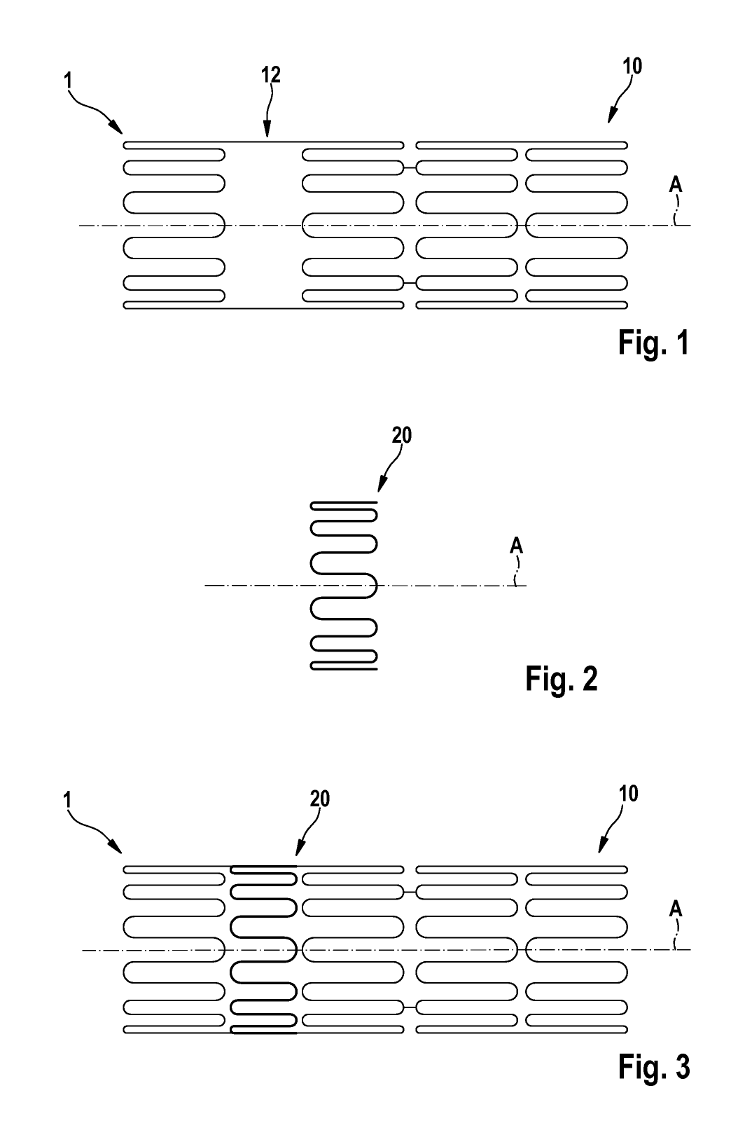

The base body 10 shown in FIG. 1 and the function element 20 shown in FIG. 2 are tubular according to an exemplary embodiment of the invention. The base body has a recess 12. The endoprosthesis 1 shown in FIG. 3 is composed of the tubular base body 10 and the function element 20, which is also tubular. The function element 20 is arranged in the recess 12 in the base body 10. The function element 20 surrounds the base body 10 at least partially and in at least partial areas, so that it is aligned concentrically with the base body 10.

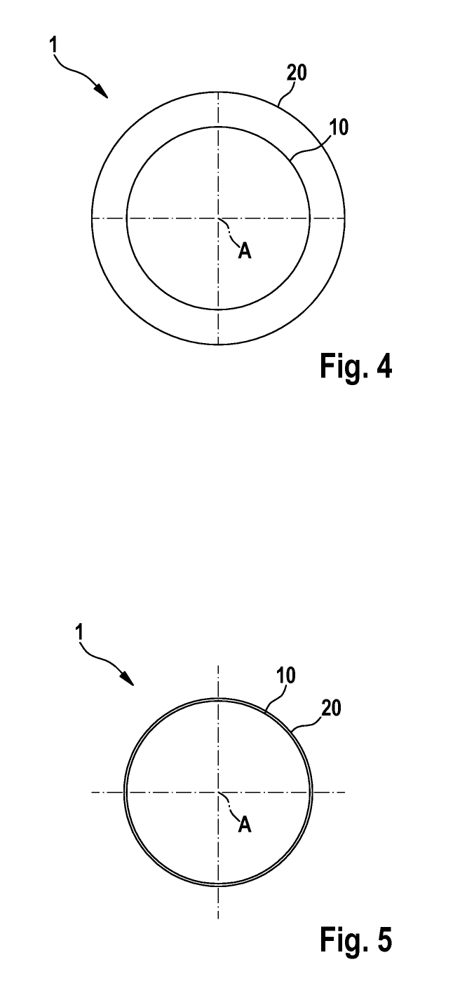

FIGS. 4 and 5 show the concentric arrangement. The base body 10 is situated on the inside and a function element 20 on the outside around the longitudinal axis A, which runs at a right angle out of the plane. In FIG. 4 the base body 10 has already been crimped, i.e., it is in a compressed form having a smaller outside diameter. In the outside area of the base body 10, the function element 20 is shown. It is arranged concentrically around the base body 10 with the common axis A. In FIG. 4 the function element 20 is as yet uncrimped and therefore it has a larger outside diameter than the base body 10.

In FIG. 5, the base body 10 and the function element 20 are equally crimped. The embodiment with recesses 12 is especially preferred. If the function element 20 fills up the recess 12 in the base body 10, as assumed here, then the base body 10 and the function element 20 have the same small diameter after crimping.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NOTATIONS

1 endoprosthesis
10 base body
12 recess
20 function element
A radial axis

What is claimed is:

1. An endoprosthesis, optionally an intraluminal endoprosthesis, comprising a tubular base body and at least one function element, wherein the at least one function element is also tubular and is arranged on the base body in such a way that the at least one function element surrounds the base body at least partially and at least in partial areas, so that it is aligned concentrically with the base body.

2. The endoprosthesis according to claim 1, wherein the base body has one or more recesses, the one or more recesses being designed so that they are finable by the function element.

3. The endoprosthesis according to claim 1, wherein the base body and/or the at least one function element has a mesh structure.

4. The endoprosthesis according to claim 3, wherein the base body and the at least one function element have the same mesh structure.

5. The endoprosthesis according to claim 1, wherein the at least one function element comprises radiopaque material.

6. The endoprosthesis according to claim 1, wherein the at least one function element contains one or more active ingredients.

7. The endoprosthesis according to claim 1, wherein the base body and/or the at least one function element has one or more coatings.

8. The endoprosthesis according to claim 1, wherein the base body is a stent.

9. The endoprosthesis according to claim 1, wherein the endoprosthesis is a stent.
